(19) United States
(12) Patent Application Publication
(10) Pub. No.: US 2021/0169903 A1
(43) Pub. Date: Jun. 10, 2021

(54) PHARMACEUTICAL COMPOSITIONS AND METHODS

(71) Applicant: Tyme, Inc., Bedminster, NJ (US)

(72) Inventors: Steven Hoffman, Mahwah, NJ (US); John Rothman, Lebanon, NJ (US)

(21) Appl. No.: 17/116,682

(22) Filed: Dec. 9, 2020

Related U.S. Application Data
(60) Provisional application No. 62/945,706, filed on Dec. 9, 2019.

(51) Int. Cl. A61K 31/575; A61K 47/10; A61K 47/34; A61K 47/26; A61K 47/18; A61K 47/22; A61K 47/14; A61P 35/00; A61K 47/20; A61K 45/06

(52) U.S. Cl. CPC A61K 31/575; A61K 47/10; A61K 47/34; A61K 47/26; A61K 45/06; A61K 47/22; A61K 47/14; A61P 35/00; A61K 47/20; A61K 47/18

(57) ABSTRACT

Methods of treating cancer or reducing tumor size by contacting a patient's cancer cells or tumor with an effective amount of a pharmaceutical composition as further defined herein.

### CROSS-REFERENCE TO RELATED APPLICATION

This application claims priority to U.S. Provisional Patent Application No. 62/945,706, filed on Dec. 9, 2019, the entirety of which is incorporated by reference herein.

### TECHNICAL FIELD

The present inventions relate generally to compositions, kits and methods for the reduction of cellular proliferation as, for example, in the treatment of cancer.

### BACKGROUND

According to the U.S. National Cancer Institute's Surveillance Epidemiology and End Results (SEER) database for the year 2008, the most recent year for which incidence data are available, 11,958,000 Americans have invasive cancers. Cancer is the second most common cause of death in the United States, behind only heart disease, and accounts for one in four deaths. It has been estimated that approximately 1600 Americans die of cancer each day. In addition to the medical, emotional and psychological costs of cancer, cancer has significant financial costs to both the individual and society. It is estimated by the National Institutes of Health that the overall costs of cancer in 2010 was $263.8 billion. In addition, it is estimated that another $140.1 billion is lost in productivity due to premature death.

Cancer treatments today include surgery, hormone therapy, radiation, chemotherapy, immunotherapy, targeted therapy, and combinations thereof. Surgical removal of cancer has advanced significantly; however, there remains a high chance of recurrence of the disease. Hormone therapy using drugs such as aromatase inhibitors and luteinizing hormone-releasing hormone analogs and inhibitors has been relatively effective in treating prostate and breast cancers. Radiation and the related techniques of conformal proton beam radiation therapy, stereotactic radiosurgery, stereotactic radiation therapy, intraoperative radiation therapy, chemical modifiers, and radio sensitizers are effective at killing cancerous cells, but can also kill and alter surrounding normal tissue. Chemotherapy drugs such as aminopterin, cisplatin, methotrexate, doxorubicin, daunorubicin and others alone and in combinations are effective at killing cancer cells, often by altering the DNA replication process. Biological response modifier (BRM) therapy, biologic therapy, biotherapy, or immunotherapy alter cancer cell growth or influence the natural immune response, and involve administering biologic agents to a patient such as an interferons, interleukins, and other cytokines and antibodies such as rituximab and trastuzumab and even cancer vaccines such as Sipuleucel-T.

Recently, new targeted therapies have been developed to fight cancer. These targeted therapies differ from chemotherapy because chemotherapy works by killing both cancerous and normal cells, with greater effects on the cancerous cells. Targeted therapies work by influencing the processes that control growth, division, and the spread of cancer cells and signals that cause cancer cells to die naturally. One type of targeted therapy includes growth signal inhibitors such as trastuzumab, gefitinib, imatinib, centuximab, dasatinib and nilotinib. Another type of targeted therapy includes angiogenesis inhibitors such as bevacizumab that inhibit cancers from increasing surrounding vasculature and blood supply. A final type of targeted therapy includes apoptosis-inducing drugs that are able to induce direct cancer cell death.

Although all of these treatments have been effective to one degree or another, they all have drawbacks and limitations. In addition to many of the treatments being expensive, they also are often too imprecise or the cancers are able to adapt to them and become resistant.

Thus, there is a great need for additional cancer treatments. In particular, there is a need for treatments for cancers that have become resistant to other forms of treatment.

### SUMMARY

The present invention provides methods of treating cancer by contacting a patient's cancer cells with a pharmaceutical composition comprising a surfactant and a bile acid or bile acid salt.

The present invention provides methods of reducing tumor size by contacting a patient's tumor with a pharmaceutical composition comprising a surfactant and a bile acid or bile acid salt.

### BRIEF DESCRIPTION OF THE FIGURES

FIG. 1 shows the average tumor volume as a function of time for vehicle (i.e. sterile water), the transdermal formulation (Surfactant 5%), and the Bile Acid Formulation (Surfactant 5%+Bile Acid 3% 3%) for the study described in Example 1.

FIG. 2 shows the mean tumor volume as a function of time post tumor implant for the groups in the study of Example 3.

FIG. 3 shows the mean body weight change as a function of time post tumor implant for the groups in the study of Example 3.

FIG. 4 shows the survival as a function of time post tumor implant for the groups in the study of Example 3.

### DETAILED DESCRIPTION OF ILLUSTRATIVE EMBODIMENTS

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder. This condition, disease or disorder can be cancer.

As employed above and throughout the disclosure the term “effective amount” refers to an amount effective, at dosages, and for periods of time necessary, to achieve the desired result with respect to the treatment of the relevant disorder, condition, or side effect. It will be appreciated that the effective amount of components of the present invention will vary from patient to patient not only with the particular compound, component or composition selected, the route of administration, and the ability of the components to elicit a desired result in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The term “stereoisomers” refers to compounds that have identical chemical constitution but differ as regards the arrangement of the atoms or groups in space.

The term “administering” means either directly administering a compound or composition of the present invention, or administering a prodrug, derivative or analog which will form an equivalent amount of the active compound or substance within the body.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, “alkyl” refers to straight chain and branched chains having the indicated number of carbon atoms, usually from 1 to 20 carbon atoms, for example 1 to 8 carbon atoms, such as 1 to 6 or 1 to 7 carbon atoms. For example, C1-6 alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “butyl” is meant to include n-butyl, sec-butyl, isobutyl and t-butyl; “propyl” includes n-propyl and isopropyl. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like.

As used herein, “alkenyl” refers to an unsaturated branched or straight-chain alkyl group having at least one carbon-carbon double bond. The group may be in either the cis or trans configuration about the double bond(s). The group may also be an aromatic group, for example, a phenyl or phenylene moiety. Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl; phenylene, and the like. In certain embodiments, an alkenyl group has from 2 to 20 carbon atoms.

As used herein, “alkynyl” refers to an unsaturated branched or straight-chain alkyl group having at least one carbon-carbon triple bond derived by the removal of two molecules of hydrogen from adjacent carbon atoms of the parent alkyl. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms.

The present disclosure is directed to methods of treating cancer in a patient in need thereof, comprising contacting the patient's cancer cells with a pharmaceutical composition comprising a surfactant and a bile acid or a salt thereof.

In some embodiments, the surfactant is an ionic surfactant.

In some embodiments, the ionic surfactant is an anionic surfactant. Anionic surfactants include, but are not limited to, alkyl sulfates, alkyl sulfonates, alkyl phosphate esters, and alkyl carboxylates, including ammonium lauryl sulfate, sodium lauryl sulfate (sodium dodecyl sulfate, SLS, or SDS), sodium tetradecyl sulfate, sodium laureth sulfate (sodium lauryl ether sulfate or SLES), sodium myreth sulfate, docusate (dioctyl sodium sulfosuccinate), perfluorooctanesulfonate (PFOS), perfluorobutanesulfonate, alkyl-aryl ether phosphates, alkyl ether phosphates, sodium lauroyl sarcosinate perfluorononanoate, perfluorooctanoate (PFOA or PFO), and ethanolamine oleate.

In other embodiments, the ionic surfactant is a cationic surfactant. Anionic surfactants include, but are not limited to linear alkyl-ammoniums, and benzalkoniums or alkyl dimethyl benzyl-ammoniums, cetrimonium bromide (CTAB), cetylpyridinium chloride (CPC), benzalkonium chloride (BAC), benzethonium chloride (BZT), dimethyldioctadecylammonium chloride, and dioctadecyldimethylammonium bromide (DODAB).

In some embodiments, the surfactant is a nonionic surfactant. Examples of nonionic surfactants include, but are not limited to, ethoxylated linear alcohols, ethoxylated alkyl phenols, fatty acid esters, and ethoxylated alkyl-amides, 2-(dodecyloxy)ethanol,

In some embodiments, the surfactant is an amphoteric (or zwitterionic) surfactant. Examples of amphoteric surfactants include, but are not limited to, sultaines CHAPS (3-[(3-cholamidopropyl)dimethylammonio]-1-propanesulfonate), cocamidopropyl hydroxysultaine; betaines such as cocamidopropyl betaine, phospholipids phosphatidylserine, phosphatidylethanolamine, phosphatidylcholine, and sphingomyelins.

Specific anionic, cationic, nonionic and amphoteric (or zwitterionic) surfactants are known to those of skill in the art. See, e.g., Salager, J-L, Surfactants Types and Uses, Laboratory of Formulation, Interfaces Rheology and Processes, Universidad De Los Andes, Merida-Venezuela, Version #2 (2002).

In some embodiments, the surfactant is a compound of formula (I)

R—(OCH₂CH₂)_{y}—OH (I)

wherein R is C₁₋₂₀alkyl, C₂₋₂₀alkenyl; or C₂₋₂₀alkynyl; and y is 1 to 25.

In some embodiments of the disclosure wherein the surfactant is a compound of formula (I), R is C₁₋₂₀alkyl, which can either be a straight chain or branched alkyl. Preferred compounds of formula I wherein R is C₁₋₂₀alkyl include, for example, is cetomacrogol 1000; octadecan-1-ol, ethoxylated; polyoxyethylene(12)tridecyl ether; polyoxyethylene(10)tridecyl ether; fatty alcohol polyoxyethylene ether, polyoxyethylene branched nonylcyclohexyl ether (TRITON N-101), nonaethylene glycol monododecyl ether, 23-{[4-(2,4,4-trimethyl-2-pentanyl)cyclohexyl]oxy}-3,6,9,12,15,18,21-heptaoxatricosan-1-ol, and combinations thereof. Nonaethylene glycol monododecyl ether is particularly preferred.

In other embodiments, R is C₂₋₂₀alkenyl, which can either be a straight chain or branched alkenyl. Preferred compounds of formula I wherein R is C₂₋₂₀alkenyl include, for example, polyoxyl(10)oleyl ether, polyethylene glycol tert-octylphenyl ether (TRITON X-100), and combinations thereof.

In yet other embodiment, R is C₂₋₂₀alkynyl, which can either be a straight chain or branch alkynyl.

In those embodiments wherein the surfactant is a compound of formula I, y is 1 to 25. In preferred embodiments, y is 5 to 15, preferably 8 to 10, with 9 being particularly preferred. In other embodiments, y is 1, 2, 3, 4, 5, 6, 7, 8, 9, 10, 11, 12, 13, 14, 15, 16, 17, 18, 19, 20, 21, 22, 23, 24, or 25.

In some embodiments, the surfactant is a tetrafunctional block copolymer surfactant terminating in primary hydroxyl groups. Such compounds are commercially available under the tradename TETRONIC and include ethylenediaminetetrakis(ethoxylate-Block-propoxylate).

In other embodiments of the disclosure, the surfactant is a sorbitan derivative, for example, polyoxyethylene sorbitan tetraoleate, 1,4-anhydro-6-O-palmitoyl-D-glucitol (sorbitan, monohexadecanoate), a polyethylene glycol sorbitan monolaurate (e.g., TWEEN 20, TWEEN 40, TWEEN 60, TWEEN 85), and combinations thereof.

In still other embodiments of the disclosure, the surfactant is a C₈₋₁₀alkyl ammonium salt, for example, methyltrialkyl(C8-C10)ammonium chloride (ADOGEN 464).

In other embodiments, the first component is a compound of formula II:

HO—(CH₂CH₂O)ₘ—C(CH₃)(C₄H₉)—C≡C—C(CH₃)(C₄H₉)—(OCH₂CH₂)ₙ—OH (II)

wherein m and n are each independently 1 to 25.

In some embodiments, the surfactant is an amide of the formula III:

R²—N(R¹)—C(O)—R³ (III)

wherein
each R¹ is independently H or C₁₋₃alkyl; and
R² and R³ are independently C₁₋₇alkyl or together with the atoms to which they are attached, form a lactam having 3 to 10 carbon atoms.

In some embodiments, of the amide of formula III, R¹ is H. In other embodiments, R¹ is methyl, ethyl, propyl, or isopropyl, with methyl being particularly preferred.

In those embodiments of formula III wherein R² and R³ are independently C₁-7alkyl, each of R² and R³ is independently methyl, ethyl, propyl, isopropyl, butyl, s-butyl, t-butyl, pentyl, hexyl, or heptyl.

Preferably, R² and R³ in formula (III), together with the atoms to which they are attached, form a lactam having 3 to 10 carbon atoms. For example, the lactam can include 3, 4, 5, 6, 7, 8, 9, or 10 carbons, which can be a part of the lactam ring or which can form exocyclic branching. Examples of preferred lactams include pyrrolidones such as 2-pyrrolidone, 1-methyl-2-pyrrolidone, 5-methyl-2-pyrrolidone, and 1-ethyl-2-pyrrolidone. Preferably, the lactam is 1-methyl-2-pyrrolidinone or 2-pyrrolidone.

In embodiments in which the compositions comprise a compound of Formula III, the compound of Formula III can comprise from about 0.01 vol. % to about 10 vol. % of the composition. In preferred embodiments, the compositions comprise from about 0.01 vol. % to about 5 vol. % of the compound of Formula III. In other embodiments, the compositions comprise from about 0.01 vol. % to about 4 vol. % of the compound of Formula III. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or about 10 vol. % of the compound of Formula III.

In some embodiments, the compositions of the disclosure can comprise from about 0.1 vol. % to about 40 vol. % of the surfactant. In preferred embodiments, the compositions comprise from about 1 vol. % to about 40 vol. % of the surfactant. In other embodiments, the compositions comprise from about 0.1 vol. % to about 5 vol. % of the surfactant. For example, the compositions can comprise about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 vol. % of the surfactant.

In some embodiments, the surfactant is an organic acid that is not a bile acid. For example, an organic acid having 1 to 25 carbon atoms, such as fatty acids and fatty acid derivatives.

In some embodiments, the surfactant is an organic acid having 1 to 25 carbon atoms that is not a bile acid. For example, organic acids for use in the disclose compositions include acetic acid, ascorbic acid, lactic acid, glycolic acid, propionic acid, and combinations thereof.

In other embodiments, the surfactant is a fatty acid. As used herein, the term “fatty acid” has its ordinary meaning as would be understood by a person of ordinary skill in the art and includes a molecule having a carboxylic group and a hydrocarbon chain. Descriptions of the number of carbon atoms in a fatty acid herein refer to the number of carbon atoms in the hydrocarbon chain of the fatty acid, irrespective of whether the hydrocarbon chain is straight or branched.

As used herein, the term “fatty acid” includes saturated fatty acids, which do not contain any double or triple bonds in the hydrocarbon chain. Saturated fatty acids include, but are not limited to propionic acid (C3) (by way of example, C3 indicates propionic acid has 3 carbon atoms in its hydrocarbon chain; the number of carbon atoms in the hydrocarbon chain of other example fatty acids is denoted in analogous fashion herein), butyric acid (C4), valeric acid (C5), caproic acid (C6), enanthic acid (C7), caprylic acid (C8), pelargonic acid (C9), capric acid (C10), undecylic acid (C11), lauric acid (C12), tridecylic acid (C13), myristic acid (C14), pentadecylic acid (C15), palmitic acid (C16), margaric acid (C17), stearic acid (C18), isostearic acid (C18), nonadecylic acid (C19), arachidic acid (C20), heneicosylic acid (C21), behenic acid (C22), tricosylic acid (C23), lignoceric acid (C24), pentacosylic acid (C25), cerotic acid (C26), heptacosylic acid (C27), montanic acid (C28), nonacocylic acid (C29), melissic acid (C30), henatriacontylic acid (C31), lacceroic acid (C32), psyllic acid (C33), geddic acid (C34), ceroplastic acid (C35) and hexatriacontylic acid (C36).

As used herein, the term “fatty acid” also includes monounsaturated fatty acids, which contain one double or triple bond in the hydrocarbon chain, and polyunsaturated fatty acids, which contain more than one double and/or triple bond in the hydrocarbon chain. Such acids include, but are not limited to the omega 3, omega 6, omega 9 fatty acids, other fatty acids such as myristoleic and palmitoleic acid and conjugated fatty acids. Examples of monounsaturated and polyunsaturated fatty acids include but are not limited to, (a) omega 3 fatty acids, such as hexadecatrienoic acid (C16:3); (by way of example, C16:3 indicates hexadecatrienoic acid has 16 carbon atoms in its hydrocarbon chain and 3 double bonds; the number of carbon atoms and double bonds in the hydrocarbon chain of other example unsaturated fatty acids is denoted in analogous fashion herein), alpha linolenic acid (C18:3) and eicosapentanoic acid (20:5), (b) omega 6 fatty acids, such as linoleic acid (18:2), docosadienoic acid (C22:2), arachidonic acid (C20:4) and tetracosatetraenoic acid (C24:5), (c) omega 9 fatty acids, such as oleic acid (C18:1), eicosenoic acid (C20:1) and nevronic acid (C24:1), and (d) conjugated fatty acids such as rumenic acid (C18:2), eleostatic acid (C18:3), and rumelenic acid (C18:3).

As used herein, the term “fatty acid” also includes branched fatty acids. Examples of branched fatty acids include, but are not limited to, monomethyl branched fatty acids, such as 14-methyl pentadecanoic acid, 6-methyl caprylic acid, 4-methyl-3-pentenoic acid, (pyroterebic acid), 2-methyl-2E-butenoic acid (tiglic acid), 2-methyl-2Z-butenoic acid (angelic acid), multimethyl branched acids, isoprenoid fatty acids (vittatalactone, all-trans-retinoic acid), branched methoxy fatty acids and hydroxy and other fatty acids such as 2-hydroxyoctanoic acid and 4-oxopentanoic acid.

In some embodiments in which the composition comprises an organic acid having 1 to 25 carbon atoms that is not a bile acid, the compositions of the disclosure can comprise from about 0.01 vol. % to about 15 vol. % of the organic acid. In some embodiment, the compositions comprise from about 1 vol % to about 15 vol % of the organic acid. In preferred embodiments, the compositions comprise from about 0.01 vol. % to about 5 vol. % of the organic acid. In other embodiments, the compositions comprise from about 0.01 vol. % to about 3 vol. % of the organic acid. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or about 15 vol. % of the organic acid.

In some embodiments, the surfactant comprises a mixture of different surfactants. In some embodiments, the surfactant comprises a mixture of the different surfactants described above.

The compositions of the invention also comprise a bile acid or a bile acid salt. In some embodiments, the second component comprises a bile acid.

In some embodiments, the bile acid is deoxycholic acid, cholic acid, glycocholic acid, taurocholic acid, tauroursodeoxycholic acid, chenodeoxycholic acid, glycochenodeoxycholic acid, taurochenodeoxycholic acid, or lithocholic acid.

In some embodiments, the bile acid is tauroursodeoxycholic acid.

In other embodiments, the second component comprises a bile acid salt.

In some embodiments, the bile acid salt is a salt of deoxycholic acid, cholic acid, glycocholic acid, taurocholic acid, tauroursodeoxycholic acid, chenodeoxycholic acid, glycochenodeoxycholic acid, taurochenodeoxycholic acid, or lithocholic acid.

In some embodiments, the bile acid salt is a salt of tauroursodeoxycholic acid.

In some embodiments, the bile acid salt is a sodium salt of tauroursodeoxycholic acid.

In other embodiments, the bile acid salt is sodium deoxycholate.

In some embodiments, the amount of bile acid or bile acid salt can comprise from about 0.01 vol. % to about 10 vol. % of the composition. In preferred embodiments, the compositions comprise from about 0.01 vol. % to about 5 vol. % of the bile acid or bile acid salt. In other embodiments, the compositions comprise from about 0.01 vol. % to about 3 vol. % of the bile acid or bile acid salt. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or about 10 vol. % of the bile acid or bile acid salt. In some embodiments, the compositions comprise about 3 vol. % of the bile acid.

In other embodiments, the compositions used in the methods of the invention further comprise a sulfoxide, for example, dimethyl sulfoxide.

In other embodiments, the compositions used in the methods of the invention further comprise a urea, for example an imidazolidinone.

In other embodiments, the compositions used in the methods of the invention further comprise ethyl acetate.

In embodiments in which the compositions comprise a sulfoxide, a urea, or ethyl acetate, the sulfoxide, urea, or ethyl acetate can comprise from about 0.01 vol. % to about 10 vol. % of the composition. In preferred embodiments, the compositions comprise from about 0.01 vol. % to about 5 vol. % of the sulfoxide, urea, or ethyl acetate. In other embodiments, the compositions comprise from about 0.01 vol. % to about 4 vol. % of the sulfoxide, a urea, or ethyl acetate. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or about 10 vol. % of the sulfoxide, a urea, or ethyl acetate.

In other embodiments, the compositions used in the methods of the invention further comprise a C₁₋₁₀ alkyl alcohol.

Alcohols for use in the compositions of the disclosure include C₁₋₁₀alkyl alcohols having at least one —OH moiety or at least two —OH moieties. For example, preferred alcohols include glycerol, propylene glycol, methanol, ethanol, isopropanol, 1-propanol, butanol, t-butanol, pentanol, 1-octanol, benzyl alcohol, methanol, ethanol, propanol, butanol, pentanol, hexanol, octanol, nonanol, decanol, tryptophol, tyrosol, and phenylethanol, and combinations thereof, with ethanol being particularly preferred.

In some embodiments in which the composition comprises a C₁₋₁₀ alkyl alcohol, the compositions of the disclosure can comprise from about 0.1 vol. % to about 99 vol. % of the C₁₋₁₀ alkyl alcohol. In some preferred embodiments, the compositions comprise from about 1 vol. % to about 50 vol. % of the C₁₋₁₀ alkyl alcohol. In other embodiments, the compositions comprise from about 0.1 vol. % to about 5 vol. % of the C₁₋₁₀ alkyl alcohol. In other preferred embodiments, the compositions comprise about 90 to about 99 vol. % of the C₁₋₁₀ alkyl alcohol. For example, the compositions can comprise about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 95, 98, or about 99 vol. % of the C₁₋₁₀ alkyl alcohol.

Compositions of the disclosure can be anhydrous. As used herein, “anhydrous” refers to compositions comprising less than 1 vol. % of water, preferably less than 0.05 vol. % or less than 0.025 vol. % of water. Methods of determining water content are known in the art.

The first component can optionally comprise water. In some embodiments, the first component can comprise up to 99 vol. % of water. In still other aspects, the first component can comprise 5, 10, 20, 30, 40, 50, 60, 70, 80, 90, 95, or 99 vol. % of water. In other embodimentts, the first component can comprise 90, 91, 92, 93, 94, 95, 96, 97, 98, or 99 vol. % of water.

Compositions of the disclosure that include water can optionally contain one or more physiologically acceptable salts. Salts for use in the compositions include, but are not limited to, sodium chloride, potassium chloride, and mixtures thereof. A preferred form of sodium chloride is bacteriostatic sodium chloride solution.

In some embodiments, the compositions of the disclosure further comprise morrhuate sodium, chromated glycerin, sclerosant formulations, laurocapram, terpenes, hydrocarbons, such as alkanes, alkenes, halogenated alkanes, squalene, squalene, and mineral oil; amines, isopropyl myristate, terpenes, terpenoids, essential oils; lipids, such as phospholipids, cyclic oligosaccharides such as cyclodextrins, amino acids and thioacyl derivatives of amino acids, alkyl amino esters and oxazolidinones, enzymes, such as papain and medicinal leech enzymes, or ketones.

In some embodiments, the compositions of the disclosure comprise any combination of A) a compound of Formula I, a tetrafunctional block copolymer surfactant, a sorbitan derivative, a C₈₋₁₀alkyl ammonium salt, a compound of Formula II, a compound of Formula III, a sulfoxide, a urea, or ethyl acetate, a C₁₋₁₀ alkyl alcohol, an organic acid having 1 to 25 carbon atoms that is not a bile acid; and B) a bile acid or bile acid salt.

In some embodiments, the compositions of the disclosure comprise about 32-40 vol. % of a compound of Formula I, a tetrafunctional block copolymer surfactant, a sorbitan derivative, a C₈₋₁₀alkyl ammonium salt, or a compound of Formula II; about 2-4 vol. % of a compound of Formula III, a sulfoxide, a urea, or ethyl acetate; about 40-50 vol. % of the C₁₋₁₀alkyl alcohol; about 6-12 vol. % of the organic acid that is not a bile acid; and about 0.01 vol. % to about 5 vol. % or a bile acid or bile acid salt.

In other embodiments, the compositions of the disclosure comprise about 3.2-4.0 vol. % of a compound of Formula I, a tetrafunctional block copolymer surfactant, a sorbitan derivative, a C₈₋₁₀alkyl ammonium salt, or a compound of Formula II; about 0.2-0.4 vol. % of a compound of Formula III, a sulfoxide, a urea, or ethyl acetate; about 4.0-96 vol. % of the C₁₋₁₀alkyl alcohol; about 0.6-1.2 vol. % of the organic acid that is not a bile acid, and about 0.01 vol. % to about 5 vol. % or a bile acid or bile acid salt.

In other embodiments, the compositions of the disclosure comprise about 0.32-0.40 vol. % of a compound of Formula I, a tetrafunctional block copolymer surfactant, a sorbitan derivative, a C₈₋₁₀alkyl ammonium salt; about 0.02-0.04 vol. % of a compound of Formula III, a sulfoxide, a urea, or ethyl acetate; about 0.40-99.5 vol. % of the C₁₋₁₀alkyl alcohol; about 0.06-0.12 vol. % of the organic acid that is not a bile acid; and about 0.01 vol. % to about 5 vol. % or a bile acid or bile acid salt.

In other embodiments, the compositions of the disclosure comprise about 4.1 vol. % of nonaethylene glycol monododecyl ether; about 0.4 vol. % of 1-methyl-2-pyrrolidone; about 93.3 vol. % of ethanol; about 0.9 vol. % of linoleic acid, and about 0.01 vol. % to about 1 vol. % or a bile acid or bile acid salt.

In other embodiments, the compositions of the disclosure comprise about 3.2 vol. % of nonaethylene glycol monododecyl ether; about 0.3 vol. % of 1-methyl-2-pyrrolidone; about 94.8 vol. % of ethanol; about 0.7 vol. % of linoleic acid, and about 0.01 vol. % to about 1 vol. % or a bile acid or bile acid salt.

In other embodiments, the compositions of the disclosure comprise about 0.32 vol. % of nonaethylene glycol monododecyl ether; about 0.03 vol. % of 1-methyl-2-pyrrolidone; about 98.6 vol. % of ethanol; and about 0.07 vol. % of linoleic acid, and about 0.01 vol. % to about 1 vol. % or a bile acid or bile acid salt.

In some embodiments, the compositions of the disclosure comprise about 3.2-4.0 vol. % of a compound of Formula I, a tetrafunctional block copolymer surfactant, a sorbitan derivative, a C₈₋₁₀alkyl ammonium salt; about 0.2-0.4 vol. % of a compound of Formula III, a sulfoxide, a urea, or ethyl acetate; about 4.0-5.0 vol. % of the C₁₋₁₀alkyl alcohol; about 0.6-1.2 vol. % of the organic acid that is not a bile acid, and about 0.01 vol. % to about 1 vol. % or a bile acid or bile acid salt; and about 80-94 vol. % water.

In other embodiments, the compositions of the disclosure comprise about 0.32-0.40 vol. % of a compound of Formula I, a tetrafunctional block copolymer surfactant, a sorbitan derivative, a C₈₋₁₀alkyl ammonium salt; about 0.02-0.04 vol. % of the a compound of Formula III, a sulfoxide, a urea, or ethyl acetate; about 0.40-0.50 vol. % of the C₁₋₁₀alkyl alcohol; about 0.06-0.12 vol. % of the organic acid that is not a bile acid, and about 0.01 vol. % to about 1 vol. % or a bile acid or bile acid salt; and about 80-98.2 vol. % water.

In other embodiments, the compositions of the disclosure comprise about 3.2 vol. % of nonaethylene glycol monododecyl ether; about 0.3 vol. % of 1-methyl-2-pyrrolidone; about 4.3 vol. % of ethanol; about 0.7 vol. % of linoleic acid, and about 0.01 vol. % to about 1 vol. % or a bile acid or bile acid salt; about 90.5 vol. % water.

In other embodiments, the compositions of the disclosure comprise about 0.32 vol. % of nonaethylene glycol monododecyl ether; about 0.03 vol. % of 1-methyl-2-pyrrolidone; about 0.43 vol. % of ethanol; about 0.07 vol. % of linoleic acid, and about 0.01 vol. % to about 1 vol. % or a bile acid or bile acid salt; about 98.2 vol. % water.

In other embodiments, the compositions of the disclosure comprise about 5 vol. % of a compound of Formula I, a tetrafunctional block copolymer surfactant, a sorbitan derivative, a C₈₋₁₀alkyl ammonium salt; about 0.5 vol. % of a compound of Formula III, a sulfoxide, a urea, or ethyl acetate; about 7 vol. % of the C₁₋₁₀alkyl alcohol; about 2 vol. % of the organic acid that is not a bile acid, and about 0.01 vol. % to about 1 vol. % or a bile acid or bile acid salt; and about 84.5 vol. % of the water.

In other embodiments, the compositions of the disclosure comprise about 3% alcohol by volume, about 3% of a bile acid by weight (dissolved in the alcohol), about 1% of a compound of Formula I, a tetrafunctional block copolymer surfactant, a sorbitan derivative, or a C₈₋₁₀alkyl ammonium salt; about 0.1% a compound of Formula III, a sulfoxide, a urea, or ethyl acetate, and QS bacteriostatic water.

In other embodiments, the compositions of the disclosure comprise about 3% benzyl alcohol by volume, about 3% Na deoxycholate by weight (dissolved in the alcohol), about 1% of 98% nonaethylene glycol monododecyl ether by volume, about 0.1% of 99.5% anhydrous 1-methyl-2-pyrrolidinone, and QS bacteriostatic water.

In another embodiment, t the compositions of the disclosure comprise 3% benzyl alcohol by volume, 3% Na deoxycholate by weight (dissolved in the alcohol), 1% of 98% nonaethylene glycol monododecyl ether by volume, 0.1% of 99.5% anhydrous 1-methyl-2-pyrrolidinone, and QS bacteriostatic water.

In other embodiments, the alcohol can be present in an amount between about 0.3% and about 30% by volume. In an embodiment, the bile acid can be present in an amount between about 0.3% and about 30% by volume. In an embodiment, the compound of Formula I, a tetrafunctional block copolymer surfactant, a sorbitan derivative, or a C₈₋₁₀alkyl ammonium salt agent can be present in an amount between about 0.1% and about 10% by volume. In an embodiment, the compound of Formula III, a sulfoxide, a urea, or ethyl acetate can be present in an amount between about 0.01% and about 1%.

The compositions of the invention can be formulated in any pharmaceutical dosage form capable of bring the composition into contact with the cancer cells. Such compositions include dosage forms for intratumoral delivery of the compositions.

According to some embodiments, administration can be by direct injection e.g., via a syringe, at the site of a tumor or neoplastic or pre-neoplastic tissue.

A composition of the present invention can be delivered in an immediate release or in a controlled release system. In one embodiment, an infusion pump may be used to administer a compound of the invention, such as one that is used for delivering chemotherapy to specific organs or tumors.

In another embodiment, a compound of the invention is administered in combination with a biodegradable, biocompatible polymeric implant, which releases the compound over a controlled period of time at a selected site. Examples of polymeric materials include polyanhydrides, polyorthoesters, polyglycolic acid, polylactic acid, polyethylene vinyl acetate, copolymers and blends thereof. In yet another embodiment, a controlled release system can be placed in proximity of the therapeutic target, thus requiring only a fraction of the systemic dose.

In some embodiments, compositions of the invention may be formulated as solutions, gels, transdermal patches, lotions, creams, sprays, mists, emulsions, or dispersions, tablets, capsules, or powders. Appropriate excipients for formulating such dosage forms are readily apparent to a person of skill in the art and include, but are not limited to, stabilizers, emulsifiers, thickeners, antimicrobials, humectants, propellants, spreading agents, polymers, and adhesives, such as pressure sensitive adhesives. In particular, excipients that may be used to form a transdermal gel include, but are not limited to, alcohols, glycols, glycerin, butylated hydroxytoluene (BHT), and water.

The methods of the present invention are directed to treating cancer in a patient by contacting the patient's cancer cells with the disclosed composition. The cancer cells may be present in the patient as individual cells or as a mass or cancer cells, or tumor. The cancer may be of any type, including, for example, non-small cell lung cancer, brain cancer, appendix cancer, biliary cancer, choleangiocarcinoma, colon cancer, germ cell tumor, glioma, neuroblastoma, prostate cancer, tongue cancer, tonsil squamous cell carcinoma, urothelial cancer, adenoid cystic carcinoma, adrenal gland tumor, amyloidosis, anal cancer, ataxia-telangiectasia, atypical mole syndrome, Beckwith Wiedemann syndrome, bile duct cancer, Birt Hogg Dube syndrome, bladder cancer, bone cancer, brain tumor, breast cancer, breast cancer in men, carcinoid tumor, carney complex, cervical cancer, colorectal cancer, ductal carcinoma, endometrial cancer, esophageal cancer, familial-adenomatous polyposis, gastric cancer, gastrontestinal stromal tumor—GIST, HER2-positive breast cancer, hereditary prostate cancer, islet cell tumor, juvenile polyposis syndrome, kaposi's sarcoma, HIV and AIDS, kidney cancer, laryngeal cancer, leukemia—acute lymphoblastic leukemia, leukemia—acute myeloid aml, leukemia—adult, leukemia—childhood, leukemia—chronic lymphocytic—CLL, leukemia—chronic myeloid—CIVIL, leukemia—acute lymphocytic (ALL), liver cancer, lobular carcinoma, lung cancer, lung cancer—small cell, lymphoma—Hodgkin's, lymphoma—non-Hodgkin's, lynch syndrome, malignant glioma, mastocytosis, melanoma, meningioma, multiple endocrine neoplasia type 1, multiple endocrine neoplasia type 2, multiple myeloma, myelodysplastic syndrome (MDS), nasopharyngeal cancer, neuroendocrine tumor, nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma (childhood), polycythemia vera, prostate cancer, renal cell cancer, retinoblastoma (childhood), salivary gland cancer, sarcoma, sarcoma—alveolar soft part and cardiac, sarcoma—Kaposi, skin cancer (non-melanoma), small bowel cancer, small intestine cancer, small intestine cancer, stomach cancer, testicular cancer, thymoma, thyroid cancer, Turcot syndrome, uterine (endometrial) cancer, vaginal cancer, Von-Hippel-Lindau syndrome, Wilms' tumor (childhood), or xeroderma pigmentosum.

In the methods of the present invention, the cancer cells are contacted with the disclosed composition. This contact may be achieved by any suitable method that brings the cancer cells and the disclosed composition into physical contact. For example, cancer cells in or at the surface of the skin may be contacted by topically applying the disclosed composition to the skin at the location of the cancer cells such that the composition comes into physical contact with the cancer cells. Cancer cells in a tumor within the patient's body may be brought into physical contact with the disclosed compositions by injecting the composition into the tumor within the patient's body. Similarly, cancer cells may be brought into contact with the disclosed compositions by applying the composition to the cancer cells during surgery. In this mode, the tumor or cancer cells are accessed during surgery and the composition is physically applied to the cells or injected into the tumor.

In other aspects, the disclosure is directed to methods of reducing the size of a tumor in a patient by contacting the patient's tumor with an effective amount of a pharmaceutical composition comprising a surfactant and a bile acid or bile acid salt as described herein.

In yet other aspects, the methods of the invention are directed to methods of treating a lesion in a patient by contacting the lesion with any of the pharmaceutical compositions as described herein. In some embodiments, the lesion is present in a tissue of the breast, prostate, lung, colon, stomach, pancreas, ovary, brain, skin, bone, fat, lymph, gastrointestinal tract, liver, or soft tissue. In other embodiments, the lesion is noncancerous.

The composition of the invention may be administered only once, or it may be administered multiple times. For multiple dosages, the composition may be, for example, administered three times a day, twice a day, once a day, once every two days, twice a week, weekly, once every two weeks, or monthly. Suitable dosage ranges and schedules can vary.

In some embodiments, a tumor in a subject is intratumorally injected on about day 1 and about day 3, on about day 8 and about day 10, on about day 15 and about day 17, on about day 22 and about day 24, on about day 29 and about day 31, and on about day 36. In some embodiments, dosing volume comprises about 50 μl of the composition per tumor during week one. In an embodiment, dosing volume comprises about 100 μl of the composition per tumor for week two. In an embodiment, dosing volume comprises about 200 μl of the composition for the remainder of the dosing administrations.

In other embodiments, dosing volume of the composition can range from about 5 μl to about 2000 μl of the composition per tumor. In other embodiments, dosing volumes can range from about 5 μl to about 500 μl of the composition per tumor. In embodiments, dosing volume of the composition can range from about 10 μl to about 1000 μl of the composition per tumor.

In other embodiments, dosing volume of the composition can range from about 20 μl to about 2000 μl of the composition per tumor. In one embodiment, dosing volume comprises 50 μl of the composition per tumor during week one, 100 μl of the composition per tumor for week two, and 200 pi of the composition for the remainder of the dosing administrations. In another embodiment, dosing volume comprises 50 μl of the composition administered on day 1 and day 3, 100 μl of the composition per tumor administered on day 8 and day 10, and 200 μl of the composition administered on day 15, day 17, day 22, day 24, day 29, day 31, and day 36. Determining other suitable dosing schedules and composition dosage ranges and amounts are within the skill of the ordinary artisan.

In some embodiments, the present invention provides methods of treating cancer comprising administering a composition as described herein in combination with one or more targeted therapies. In one embodiment, an immunotherapeutic compound is targeted to particular molecules expressed abnormally by cancer cells. In one embodiment, the targeted therapy comprises a hormone therapy, signal transduction inhibitor, gene expression modulator, apoptosis inducer, angiogenesis inhibitor, immunotherapy, or toxin delivery molecules. In one embodiment, the targeted therapy utilizes small molecules. In another embodiment, the targeted therapy utilizes antibodies, which, in one embodiment, are monoclonal antibodies.

In some embodiments, the methods of treating cancer of the invention further comprise administering a second chemotherapeutic agent. In some embodiments, the second chemotherapeutic agent is 2-methoxyestradiol, 3,3′-diindolylmethane, abexinostat, aceglatone, actinomycin, acutissimin Aa, afatinib, aflibercept, alemtuzumab, alestramustine, alitretinoin, all-trans retinoic acid, altretamine, aminolevulinic acid, amphinex, amsacrine, anagrelide, angiozyme, anthramycin, antibody-drug conjugate, anticarcinogen, apaziquone, APG101, arsenic trioxide, asparaginase, atrimustine, axitinib, azacitidine, azaserine, azathioprine, bcg vaccine, bendamustine, bevacizumab, bexarotene, biricodar, bleomycin, bortezomib, bosutinib, busulfan, buthionine sulfoximine, cabazitaxel, cabozantinib, capecitabine, carboplatin, carmofur, carmustine, celecoxib, cetuximab, chlorambucil, chlormethine, cisplatin, cladribine, clofarabine, copanlisib, crizotinib, crotogoudin, cyclophosphamide, cytarabine, cytestrol acetate, dacarbazine, dactinomycin, dasatinib, daunorubicin, decitabine, denileukin diftitox, denosumab, docetaxel, doxifluridine, doxorubicin, duocarmycin, efaproxiral, elsamitrucin, enasidenib, enocitabine, epirubicin, epothilone, eribulin, erlotinib, estradiol mustard, estromustine, etoglucid, etoposide, everolimus, exisulind, floxuridine, fluasterone, fludarabine, fluorouracil, fosfestrol, fotemustine, gefitinib, gemcitabine, gemtuzumab ozogamicin, histone deacetylase inhibitor, hn3 (nitrogen mustard), hydroxycarbamide, hydroxyurea, hypomethylating agent, ibritumomab tiuxetan, ici-85966, idarubicin, idelalisib, ifosfamide, imatinib, imiquimod, immunoconjugate, ingenol mebutate, ipilimumab, irinotecan, isotretinoin, ixabepilone, kedarcidin, lapatinib, lenalidomide, lomustine, lonidamine, losoxantrone, LS-1727, lucanthone, lurtotecan, masoprocol, mechlorethamine, melengestrol, melengestrol acetate, melphalan, mercaptopurine, metastasis suppressor, methotrexate, methyl aminolevulinate, methylepitiostanol, miltefosine, mitoguazone, mitomycin, mitomycin c, mitotane, mitoxantrone, mtor inhibitors, nedaplatin, nelarabine, neocarzinostatin, nilotinib, nivolumab, o6-benzylguanine, ofatumumab, onapristone, OSU-03012, oxaliplatin, paclitaxel, panitumumab, panobinostat, pazopanib, pegaspargase, pegdinetanib, pembrolizumab, pemetrexed, pentostatin, pertuzumab, phenestrol, picibanil, polymer-drug conjugates, pomalidomide, ponatinib, prednimustine, procarbazine, quisinostat, raltitrexed, regorafenib, resimmune, resiquimod, ribonucleotide reductase inhibitor, rituximab, romidepsin, ruxolitinib, selumetinib, signal transduction inhibitor, sivifene, sorafenib, SRC inhibitor, steganacin, streptozotocin, sturamustine, sunitinib, tamibarotene, tegafur, temozolomide, temsirolimus, teniposide, tesmilifene, testifenon, thalidomide, tioguanine, topotecan, tositumomab, trabectedin, trastuzumab, tretinoin, trifluridine, trifluridine/tipiracil, tyrosine hydroxylase inhibitors, vadimezan, valproate, valrubicin, vandetanib, vapreotide, vemurafenib, vinblastine, vincristine, vindesine, vinflunine, vinorelbine, or vorinostat.

In other embodiments, a tyrosine hydroxylase inhibitor is also administered.

In some embodiments, the tyrosine hydroxylase inhibitor is one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OME HCl, H-3,5-diiodo-tyr-OMe HCl, H-D-3,5-diiodo-tyr-OMe HCl, H-D-tyr-OMe HCl, D-tyrosine methyl ester hydrochloride, D-tyrosine-OMe HCl, methyl D-tyrosinate hydrochloride, H-D-tyr-OMe.HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)-OSu, Fmoc-tyr(3—NO₂)—H, α-methyl-DL-tyrosine, α-methyl-D-tyrosine, α-methyl-L-tyrosine, and C₁-C₁₂ alkylester salts of α-methyl-DL-tyrosine such as α-methyl-DL-tyrosine methyl ester hydrochloride.

In some embodiments, the tyrosine hydroxylase inhibitor is α-methyl-DL-tyrosine.

In other embodiments, the subject is also administered a combination of a tyrosine hydroxylase inhibitor, melanin and/or a melanin promoter, a p450 3A4 promoter, and a leucine aminopeptidase inhibitor.

In some embodiments, the second chemotherapeutic agent is a growth hormone inhibitor, such as octreotide.

The second chemotherapeutic agent or tyrosine hydroxylase inhibitor may be administered in any suitable dosage form, including tablets, capsules, caplets, sterile aqueous or organic solutions, reconstitutable powders, elixirs, liquids, colloidal or other types of suspensions, emulsions, beads, beadlets, granules, microparticles, nanoparticles, and combinations thereof. The amount of second chemotherapeutic agent administered will, of course, be dependent on the subject being treated, the subject's weight, the severity of the condition being treated, the manner of administration, and the judgment of the prescribing physician.

The second chemotherapeutic agent or tyrosine hydroxylase inhibitor may be administered through any suitable route, including orally, nasally, subcutaneously, intravenously, intramuscularly, transdermally, vaginally, rectally or in any combination thereof.

In other embodiments, the compositions, formulations, and methods described herein can comprise the inclusion or use of a pain-reducing agent in an amount effective to reduce pain in the subject. In some embodiments, the pain reducing agent comprises a general anesthetic. In other embodiments, the pain reducing agent comprises a local anesthetic. In other embodiments, the pain reducing agent comprises lidocaine. In other embodiments, the composition comprises about 1% of 2% lidocaine. In another embodiment, the composition comprises 1% of 2% lidocaine. In some embodiments, the pain reducing agent can be present in any preparation suitable for use in accordance with the compositions and methods described herein, including, without limitation, 0.5%, 1%, 1.5%, 2%, 4%, or 5% injectable solution; or a 200,400, or 800 mg/mL preparation. In an embodiment, the composition comprises between about 0.1% and about 1% of a pain reducing agent. In an embodiment, the composition comprises between about 1% and about 10% of a pain reducing agent.

In an embodiment, the pain reducing agent is comprised within the tumor reducing composition. In an embodiment, the pain reducing agent is administered separately from the tumor reducing composition.

Other suitable pain reducing agents for use in accordance with the present invention include, without limitation, procaine, bupivacaine, mepivacine, chloroprocine, tetracaine, ropivacaine, benzocaine, or any other suitable pain reducing agent known to one of ordinary 20 skill in the art.

The present methods can include not only the disclosed administration step but also the step of assessing progression of said cancer in said subject and/or the extent of cellular proliferation. The assessing step can be performed before or after the administering step.

Also provided herein are kits comprising a composition of the disclosure together with packaging for same. The kit can further comprise a second chemotherapeutic agent.

Methods of reducing cell proliferation in a subject are also provided comprising by contacting the patient's cancer cells with the disclosed composition.

In some embodiments, the method further comprises administering a second therapeutic agent.

The following examples of specific embodiments for carrying out the present invention are offered for illustrative purposes only and are not intended to limit the scope of the present invention in any way.

Representative methods of administration of the pharmaceutical compositions and combination therapies also are provided. Various embodiments of the present invention further relate to methods of administering a pharmaceutical composition or combination therapy to a human patient for the treatment of cancer. The methods may comprise administering a pharmaceutical composition or combination therapy by generally accepted routes of administration (e.g., oral, subcutaneous, parenteral, inhalation, topical, etc.). In some instances, a pharmaceutical composition or combination therapy may be administered orally and/or subcutaneously. In some instances, a pharmaceutical composition or combination therapy may be administered to human patients between meals.

In certain embodiments of the present invention, a pharmaceutical composition or combination therapy may be administered to a human patient for 5 days per week for a period of 6 weeks, creating one cycle of 30 days of treatment. Depending on the outcome after 6 weeks or one cycle of treatment, additional cycles of the pharmaceutical composition or combination therapy may be administered.

### Example 1

Efficacy Study in CT26 Tumor Model

36 female CB17 SCID mice (6-7 weeks old, acclimated for 5 days) were implanted with CT26 tumor cells on day 0.

Tumors were injected with either sterile water, formulation Surfactant 5%, or formulation Surfactant 5%+Bile Acid 3% on days 6, 9, 12, 15, 18, and 21 at a dose of 50μL per tumor for first four injections, and 100 μL per tumor for last two injections. (The first dosing of mouse #10, 11, 12 and mouse #22, 23, 24 was on 11/9, thus these tumors were treated 5 times.)

Composition Surfactant 5% is prepared by (1) mixing 3 mL of Polidocanol (100% purity), 0.3 mL of N-methylpyrrolidone (NMP; 99.5% purity), 1 mL linoleic acid (67% purity), and 4 mL of ethanol (100%); and (2) diluting the resulting mixture with water to give a mixture of 5% of the surfactant mixture and 95% water.

Composition Surfactant 5%+Bile Acid 3% comprises 5% of the surfactant mixture as prepared above, 3% of the bile acid tauroursodeoxycholic acid (TDUCA), and 92% water.

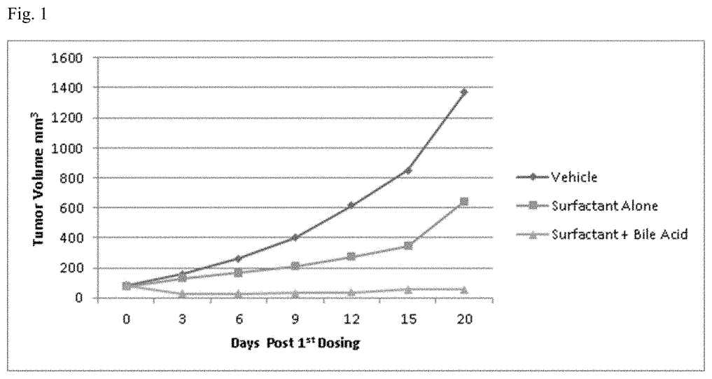

Tumors volume is measured by inspection. Tables 1-1, 1-2, and 1-3 below show the results of this study. Table 1-1 shows the tumor volume data for each individual mouse. Table 1-2 provides the average tumor volume in each group. This study demonstrates that the formulations Surfactant 5% and Surfactant 5%+Bile Acid 3% both slow tumor growth relative to sterile water, with the Surfactant 5%+Bile Acid 3% mixture slowing tumor growth most. See also FIG. 1. Table 1-3 shows the number of tumor free mice by treatment group.

TABLE 1-1

| | | | Tumor | | | | | | | |
|---|---|---|---|---|---|---|---|---|---|---|
| | | | dimensions | | Vol. | | Vol. | | Vol. | |
| Group | Treatment | Mouse ID | (mm) | Day 6 | (mm³) | Day 9 | (mm³) | Day 12 | (mm³) | Day 15 |
| 1 | Sterile | 1 | Tumor Length | 6.83 | 129 | 8.6 | 292 | 10.88 | 492 | 12.85 |
| | Water | | Tumor Width | 6.14 | | 8.24 | | 9.51 | | 10.99 |
| | | 2 | Tumor Length | 6.09 | 112 | 8.35 | 217 | 8.26 | 251 | 9.22 |
| | | | Tumor Width | 6.06 | | 7.21 | | 7.79 | | 7.71 |
| | | 3 | Tumor Length | 5.7 | 72 | 8.3 | 204 | 10.24 | 309 | 12 |
| | | | Tumor Width | 5.03 | | 7.01 | | 7.77 | | 9.18 |
| | | 4 | Tumor Length | 6.09 | 97 | 7.93 | 178 | 10.83 | 454 | 11.12 |
| | | | Tumor Width | 5.63 | | 6.7 | | 9.16 | | 10.11 |
| | | 5 | Tumor Length | 7.1 | 66 | 7.11 | 70 | 8.33 | 101 | 8.85 |
| | | | Tumor Width | 4.31 | | 4.45 | | 4.92 | | 4.94 |
| | | 6 | Tumor Length | 6.08 | 60 | 7.47 | 129 | 7.25 | 122 | 7.27 |
| | | | Tumor Width | 4.43 | | 5.87 | | 5.79 | | 5.76 |
| | | 7 | Tumor Length | 7.25 | 122 | 8.3 | 224 | 10.45 | 392 | 12.08 |
| | | | Tumor Width | 5.81 | | 7.35 | | 8.66 | | 10.08 |
| | | 8 | Tumor Length | 5.55 | 61 | 8.64 | 188 | 10.16 | 319 | 10.99 |
| | | | Tumor Width | 4.68 | | 6.59 | | 7.92 | | 7.96 |
| | | 9 | Tumor Length | 7.01 | 86 | 8.49 | 171 | 9.95 | 240 | 11.42 |
| | | | Tumor Width | 4.94 | | 6.34 | | 6.95 | | 7.49 |
| | | 10 | Tumor Length | 4.07 | 32 | 5.32 | 50 | 6.04 | 76 | 6.72 |
| | | | Tumor Width | 3.95 | | 4.33 | | 5.03 | | 6.09 |
| | | 11 | Tumor Length | 5.62 | 35 | 6.28 | 57 | 9.48 | 125 | 11.82 |
| | | | Tumor Width | 3.52 | | 4.25 | | 5.14 | | 6.6 |
| | | 12 | Tumor Length | 6.44 | 38 | 7.53 | 68 | 10.93 | 176 | 12.61 |
| | | | Tumor Width | 3.43 | | 4.24 | | 5.67 | | 7.12 |
| | | 1 | Tumor Length | 6.4 | 96 | 7.67 | 153 | 8.96 | 332 | 12.05 |
| | | | Tumor Width | 5.47 | | 6.31 | | 8.61 | | 8.86 |
| | | 2 | Tumor Length | 8.5 | 99 | 9.4 | 217 | 10.7 | 223 | 12.41 |
| | | | Tumor Width | 4.83 | | 6.79 | | 6.46 | | 7.86 |
| | | 3 | Tumor Length | 5.91 | 86 | 7.86 | 219 | 9.94 | 416 | 12.15 |
| | | | Tumor Width | 5.41 | | 7.46 | | 9.15 | | 11.52 |
| | | 4 | Tumor Length | 6.79 | 98 | 9.16 | 116 | 11.17 | 235 | 12.94 |
| | | | Tumor Width | 5.36 | | 5.03 | | 6.49 | | 7.31 |
| | | 5 | Tumor Length | 6.68 | 65 | 7.11 | 69 | 8.36 | 117 | 9.93 |
| | | | Tumor Width | 4.42 | | 4.42 | | 5.29 | | 5.66 |
| | | 6 | Tumor Length | 6.94 | 103 | 6.85 | 116 | 9.37 | 219 | 11.72 |
| | | | Tumor Width | 5.44 | | 5.83 | | 6.84 | | 8.45 |
| | | 7 | Tumor Length | 6.95 | 130 | 8.65 | 295 | 9.79 | 431 | 11.54 |
| | | | Tumor Width | 6.11 | | 8.26 | | 9.38 | | 11.02 |
| | | 8 | Tumor Length | 8.92 | 76 | 10.66 | 126 | 12.16 | 238 | 14.54 |
| | | | Tumor Width | 4.14 | | 4.87 | | 6.26 | | 7.47 |
| | | | Group Avg STD | | 83 | | 158 | | 263 | |
| | | | p value (t-test) | | 30 | | 75 | | 126 | |
| 2 | Surfactant | 13 | Tumor Length | 7.48 | 130 | 10.61 | 283 | 11.97 | 360 | 12.65 |
| | 5% | | Tumor Width | 5.9 | | 7.3 | | 7.76 | | 8.66 |
| | | 14 | Tumor Length | 7.45 | 104 | 8.16 | 210 | 8.46 | 240 | 8.18 |
| | | | Tumor Width | 5.29 | | 7.17 | | 7.53 | | 7.66 |
| | | 15 | Tumor Length | 6.03 | 78 | 8.36 | 60 | 8.46 | 118 | 8.64 |
| | | | Tumor Width | 5.09 | | 3.78 | | 5.29 | | 5.22 |
| | | 16 | Tumor Length | 5.69 | 90 | 6.32 | 94 | 6.29 | 99 | 5.91 |
| | | | Tumor Width | 5.64 | | 5.46 | | 5.6 | | 4.94 |
| | | 17 | Tumor Length | 5.2 | 63 | 6.86 | 91 | 6.5 | 90 | 6.61 |
| | | | Tumor Width | 4.92 | | 5.14 | | 5.25 | | 5.74 |
| | | 18 | Tumor Length | 8.91 | 116 | 9.47 | 245 | 10.42 | 242 | 10.34 |
| | | | Tumor Width | 5.1 | | 7.2 | | 6.81 | | 7.55 |
| | | 19 | Tumor Length | 6.03 | 83 | 8.09 | 107 | 8.46 | 142 | 8.57 |
| | | | Tumor Width | 5.24 | | 5.15 | | 5.79 | | 5.96 |
| | | 20 | Tumor Length | 6.5 | 116 | 8.22 | 214 | 10.55 | 335 | 12.24 |
| | | | Tumor Width | 5.98 | | 7.21 | | 7.97 | | 8.63 |
| | | 21 | Tumor Length | 7.25 | 38 | 6.42 | 42 | 6.87 | 59 | 7.06 |
| | | | Tumor Width | 3.24 | | 3.6 | | 4.14 | | 4.31 |
| | | 22 | Tumor Length | 4.65 | 36 | 7.06 | 94 | 7.83 | 117 | 8.5 |
| | | | Tumor Width | 3.95 | | 5.17 | | 5.47 | | 6.66 |
| | | 23 | Tumor Length | 3.82 | 26 | 4.93 | 50 | 6.06 | 62 | 6.44 |
| | | | Tumor Width | 3.69 | | 4.52 | | 4.54 | | 5.14 |
| | | 24 | Tumor Length | 4.44 | 36 | 7.11 | 83 | 8.75 | 131 | 10.82 |
| | | | Tumor Width | 4 | | 4.83 | | 5.48 | | 6.93 |
| | | | Group Avg | | 76 | | 131 | | 166 | |
| | | | STD | | 36 | | 83 | | 103 | |
| | | | p value (t-test) | | 0.597675 | | 0.368738 | | 0.025059 | |
| 3 | Surfactant 5% + | 9 | Tumor Length | 5.51 | 80 | 0 | 0 | 0 | 0 | 0 |
| | Bile Acid 3% | | Tumor Width | 5.4 | | 0 | | 0 | | 0 |
| | | 10 | Tumor Length | 6.28 | 97 | 8.61 | 155 | 11.98 | 207 | 12.24 |
| | | | Tumor Width | 5.56 | | 6 | | 5.88 | | 8.03 |
| | | 11 | Tumor Length | 6.99 | 125 | 0 | 0 | 0 | 0 | 0 |
| | | | Tumor Width | 5.98 | | 0 | | 0 | | 0 |
| | | 12 | Tumor Length | 7.55 | 55 | 0 | 0 | 0 | 0 | 0 |
| | | | Tumor Width | 3.8 | | 0 | | 0 | | 0 |
| | | 13 | Tumor Length | 7.52 | 48 | 4.23 | 19 | 6.16 | 47 | 0 |
| | | | Tumor Width | 3.56 | | 3.01 | | 3.9 | | 0 |
| | | 14 | Tumor Length | 5.7 | 47 | 4.27 | 35 | 4.38 | 40 | 0 |
| | | | Tumor Width | 4.08 | | 4.06 | | 4.27 | | 0 |
| | | 15 | Tumor Length | 7.62 | 54 | 0 | 0 | 4.07 | 9 | 0 |
| | | | Tumor Width | 3.77 | | 0 | | 2.06 | | 0 |
| | | 16 | Tumor Length | 6.11 | 89 | 4.72 | 37 | 0 | 0 | 0 |
| | | | Tumor Width | 5.39 | | 3.95 | | 0 | | 0 |
| | | 17 | Tumor Length | 7.5 | 95 | 5.28 | 31 | 5.06 | 35 | 0 |
| | | | Tumor Width | 5.04 | | 3.43 | | 3.73 | | 0 |
| | | 18 | Tumor Length | 8.81 | 75 | 3.09 | 10 | 4.23 | 10 | 4.58 |
| | | | Tumor Width | 4.13 | | 2.56 | | 2.14 | | 2.64 |
| | | 19 | Tumor Length | 6.76 | 114 | 3.26 | 18 | 0 | 0 | 0 |
| | | | Tumor Width | 5.8 | | 3.32 | | 0 | | 0 |
| | | | Tumor Length | 8.19 | | 4.93 | 33 | 0 | 0 | 0 |
| | | 20 | Tumor Width | 4.61 | 87 | 3.68 | | 0 | | 0 |
| | | | Group Avg | | 81 | | 28 | | 29 | |
| | | | STD | | 26 | | 43 | | 59 | |
| | | | p value (t-test) | | 0.799863 | | 7E−07 | | 8.04E−08 | |

| | | | Tumor | | | | | | | |
|---|---|---|---|---|---|---|---|---|---|---|
| | | | dimensions | Vol. | | Vol. | | Vol. | | Vol. |
| Group | Treatment | Mouse ID | (mm) | (mm³) | Day 18 | (mm³) | Day 21 | (mm³) | Day 26 | (mm³) |
| 1 | Sterile | 1 | Tumor Length | 776 | 15.12 | 1098 | 15.47 | 1331 | 19.25 | 2000 |
| | Water | | Tumor Width | | 12.05 | | 13.12 | | 15.99 | |
| | | 2 | Tumor Length | 274 | 9.03 | 338 | 9.65 | 364 | 11.24 | 696 |
| | | | Tumor Width | | 8.65 | | 8.68 | | 11.13 | |
| | | 3 | Tumor Length | 506 | 16.22 | 1065 | 16.34 | 1242 | 21.39 | 2000 |
| | | | Tumor Width | | 11.46 | | 12.33 | | 17.34 | |
| | | 4 | Tumor Length | 568 | 12.64 | 870 | 12.86 | 1000 | 17.18 | 2000 |
| | | | Tumor Width | | 11.73 | | 12.47 | | 15.96 | |
| | | 5 | Tumor Length | 108 | 9.98 | 173 | 10.63 | 230 | 13.7 | 502 |
| | | | Tumor Width | | 5.88 | | 6.58 | | 8.56 | |
| | | 6 | Tumor Length | 121 | 6.03 | 81 | 5.47 | 75 | 6.08 | 86 |
| | | | Tumor Width | | 5.17 | | 5.25 | | 5.31 | |
| | | 7 | Tumor Length | 614 | 13.84 | 1059 | 14.51 | 1222 | 18.46 | 2000 |
| | | | Tumor Width | | 12.37 | | 12.98 | | 16.59 | |
| | | 8 | Tumor Length | 348 | 12.39 | 530 | 13.1 | 633 | 15.45 | 1270 |
| | | | Tumor Width | | 9.25 | | 9.83 | | 12.82 | |
| | | 9 | Tumor Length | 320 | 13.11 | 533 | 15.15 | 848 | 16.74 | 1436 |
| | | | Tumor Width | | 9.02 | | 10.58 | | 13.1 | |
| | | 10 | Tumor Length | 125 | 9.58 | 235 | 9.57 | 299 | 14.05 | 916 |
| | | | Tumor Width | | 7 | | 7.9 | | 11.42 | |
| | | 11 | Tumor Length | 257 | 15.29 | 622 | 15.99 | 677 | 21.39 | 1608 |
| | | | Tumor Width | | 9.02 | | 9.2 | | 12.26 | |
| | | 12 | Tumor Length | 320 | 14.72 | 538 | 15.43 | 649 | 18.23 | 1463 |
| | | | Tumor Width | | 8.55 | | 9.17 | | 12.67 | |
| | | 1 | Tumor Length | 473 | 13.86 | 574 | 18.44 | 1028 | 20.14 | 1212 |
| | | | Tumor Width | | 9.1 | | 10.56 | | 10.97 | |
| | | 2 | Tumor Length | 383 | 13.72 | 543 | 16.27 | 968 | 17.93 | 1675 |
| | | | Tumor Width | | 8.9 | | 10.91 | | 13.67 | |
| | | 3 | Tumor Length | 806 | 14.5 | 1343 | 16.94 | 2000 | 19.56 | 2000 |
| | | | Tumor Width | | 13.61 | | 15.83 | | 17.71 | |
| | | 4 | Tumor Length | 346 | 14.97 | 547 | 17.54 | 1081 | 19.64 | 1616 |
| | | | Tumor Width | | 8.55 | | 11.1 | | 12.83 | |
| | | 5 | Tumor Length | 159 | 10.41 | 212 | 12.97 | 490 | 15.02 | 766 |
| | | | Tumor Width | | 6.38 | | 8.69 | | 10.1 | |
| | | 6 | Tumor Length | 418 | 11.79 | 452 | 14.14 | 855 | 15.65 | 1380 |
| | | | Tumor Width | | 8.76 | | 11 | | 13.28 | |
| | | 7 | Tumor Length | 701 | 12.08 | 790 | 12.61 | 828 | 13.21 | 852 |
| | | | Tumor Width | | 11.44 | | 11.46 | | 11.36 | |
| | | 8 | Tumor Length | 406 | 16.27 | 677 | 17.95 | 1168 | 19.19 | 1899 |
| | | | Tumor Width | | 9.12 | | 11.41 | | 14.07 | |
| | | | Group Avg STD | 401 | | 614 | | 849 | | 1369 |
| | | | p value (t-test) | 210 | | 339 | | 450 | | 572 |
| 2 | Surfactant | 13 | Tumor Length | 474 | 15.11 | 642 | 16.44 | 943 | 20.12 | 1740 |
| | 5% | | Tumor Width | | 9.22 | | 10.71 | | 13.15 | |
| | | 14 | Tumor Length | 240 | 7.75 | 176 | 8.26 | 213 | 9.65 | 332 |
| | | | Tumor Width | | 6.73 | | 7.18 | | 8.3 | |
| | | 15 | Tumor Length | 118 | 7.47 | 103 | 7.26 | 102 | 5.33 | 47 |
| | | | Tumor Width | | 5.24 | | 5.3 | | 4.22 | |
| | | 16 | Tumor Length | 72 | 3.73 | 15 | 2.24 | 5 | 0.87 | 0 |
| | | | Tumor Width | | 2.85 | | 2.12 | | 0.71 | |
| | | 17 | Tumor Length | 109 | 8.83 | 228 | 8.92 | 312 | 13.63 | 1154 |
| | | | Tumor Width | | 7.19 | | 8.36 | | 13.01 | |
| | | 18 | Tumor Length | 295 | 9.62 | 361 | 9.11 | 309 | 4.89 | 71 |
| | | | Tumor Width | | 8.66 | | 8.24 | | 5.38 | |
| | | 19 | Tumor Length | 152 | 8.02 | 159 | 7.31 | 144 | 9.8 | 326 |
| | | | Tumor Width | | 6.29 | | 6.28 | | 8.16 | |
| | | 20 | Tumor Length | 456 | 12.28 | 477 | 11.21 | 419 | 11.43 | 500 |
| | | | Tumor Width | | 8.81 | | 8.65 | | 9.35 | |
| | | 21 | Tumor Length | 66 | 7.19 | 78 | 4.72 | 48 | 6.68 | 119 |
| | | | Tumor Width | | 4.67 | | 4.51 | | 5.98 | |
| | | 22 | Tumor Length | 189 | 10.67 | 330 | 11.91 | 480 | 16.28 | 1099 |
| | | | Tumor Width | | 7.86 | | 8.98 | | 11.62 | |
| | | 23 | Tumor Length | 85 | 6.93 | 147 | 7.88 | 212 | 9.32 | 325 |
| | | | Tumor Width | | 6.51 | | 7.33 | | 8.35 | |
| | | 24 | Tumor Length | 260 | 13.9 | 577 | 16.01 | 962 | 20.97 | 2000 |
| | | | Tumor Width | | 9.11 | | 10.96 | | 14.32 | |
| | | | Group Avg | 210 | | 274 | | 346 | | 643 |
| | | | STD | 141 | | 203 | | 317 | | 688 |
| | | | p value (t-test) | 0.004393 | | 0.00132 | | 0.000878 | | 0.005965 |
| 3 | Surfactant 5% + | 9 | Tumor Length | 0 | 0 | 0 | 0 | 0 | 0 | 0 |
| | Bile Acid 3% | | Tumor Width | | 0 | | 0 | | 0 | |
| | | 10 | Tumor Length | 395 | 12.14 | 429 | 14.22 | 653 | 14.55 | 686 |
| | | | Tumor Width | | 8.41 | | 9.58 | | 9.71 | |
| | | 11 | Tumor Length | 0 | 0 | 0 | 0 | 0 | 0 | 0 |
| | | | Tumor Width | | 0 | | 0 | | 0 | |
| | | 12 | Tumor Length | 0 | 0 | 0 | 0 | 0 | 0 | 0 |
| | | | Tumor Width | | 0 | | 0 | | 0 | |
| | | 13 | Tumor Length | 0 | 0 | 0 | 0 | 0 | 0 | 0 |
| | | | Tumor Width | | 0 | | 0 | | 0 | |
| | | 14 | Tumor Length | 0 | 0 | 0 | 0 | 0 | 0 | 0 |
| | | | Tumor Width | | 0 | | 0 | | 0 | |
| | | 15 | Tumor Length | 0 | 0 | 0 | 0 | 0 | 0 | 0 |
| | | | Tumor Width | | 0 | | 0 | | 0 | |
| | | 16 | Tumor Length | 0 | 0 | 0 | 0 | 0 | 0 | 0 |
| | | | Tumor Width | | 0 | | 0 | | 0 | |
| | | 17 | Tumor Length | 0 | 0 | 0 | 0 | 0 | 0 | 0 |
| | | | Tumor Width | | 0 | | 0 | | 0 | |
| | | 18 | Tumor Length | 16 | 0 | 0 | 0 | 0 | 0 | 0 |
| | | | Tumor Width | | 0 | | 0 | | 0 | |
| | | 19 | Tumor Length | 0 | 0 | 0 | 0 | 0 | 0 | 0 |
| | | | Tumor Width | | 0 | | 0 | | 0 | |
| | | | Tumor Length | 0 | 0 | 0 | 0 | 0 | 0 | 0 |
| | | 20 | Tumor Width | | 0 | | 0 | | 0 | |
| | | | Group Avg | 34 | | 36 | | 54 | | 57 |
| | | | STD | 114 | | 124 | | 188 | | 198 |
| | | | p value (t-test) | 4.64E−07 | | 2.46E−07 | | 1.57E−07 | | 3.24E−05 |

TABLE 1-2

| | | Day (post 1st dosing) | | | | | | |
|---|---|---|---|---|---|---|---|---|
| Group | | 0 | 3 | 6 | 9 | 12 | 15 | 20 |
| Group 1, | Group Avg | 83 | 158 | 263 | 401 | 614 | 849 | 1369 |
| Vehicle | STD | 30 | 75 | 126 | 210 | 339 | 450 | 572 |
| Group 2, | Group Avg | 76 | 131 | 166 | 210 | 274 | 346 | 643 |
| Surfactant 5% | STD | 36 | 83 | 103 | 141 | 203 | 317 | 688 |
| Group 3, Surfactant | Group Avg | 81 | 28 | 29 | 34 | 36 | 54 | 57 |
| 5% + Bile Acid 3% | STD | 26 | 43 | 59 | 114 | 124 | 188 | 198 |

TABLE 1-3
Tumor Free Mice

| | | # of | # Tumor Free | % Tumor Free |
|---|---|---|---|---|
| Group # | Treatment | Mice | Mice | Mice |
| 1 | Vehicle | 20 | 0 | 0 |
| 2 | Surfactant Alone | 12 | 1 | 8.33 |
| 3 | Tyme-18 | 12 | 11 | 91.6 |

### Example 2

A study of 12 mice in which CT26 tumors were implanted was conducted as follows.

The study included a control group of 6 mice which received a control injection of bacteriostatic water and an experimental group of 6 mice which received the experimental formulation.

The experimental formulation was comprised of:
3% benzyl alcohol by volume;
3% Na deoxycholate by weight (dissolved in the alcohol);
1% of 98% nonaethylene glycol monododecyl ether by volume;
0.1% of 99.5% anhydrous 1-methyl-2-pyrrolidinone; and
QS with bacteriostatic water

After an acclimation period of 3-5 days, 15 BalB/C female mice were inoculated in the right flank with 1 million of CT26 cells (suspended in 100 μl 1× Bacteriostatic H₂O).

Beginning at Day 4 post cell inoculation, the tumor volume was measured every day until their average volume reached 100 mm³ (Volume=length×width×width×0.52).

Twelve (12) tumor bearing mice with preferred tumor volume were selected and randomly grouped into 2 groups (n=6 per group) and individually identified (tail mark or ear tag).

Mice were weighed and intratumorally injected with 1× Bacteriostatic H₂O or the experimental formulation on Day 1 and 3, then on Day 8 and 10, Day 15 and 17, Day 22 and 24, Day 29 and 31, and Day 36.

The dosing volume of 1× Bacteriostatic H₂O or experimental formulation was at 50 μl each tumor for first week, 100 μl for each tumor for 2nd week, 200 μl per tumor for the rest of dosing.

Tumor volume and body weight of mice were measured twice a week until the termination of the study or the group average tumor volume of the control group reached 1500-2000 mm³ at which the study was be terminated.

At the end of a 3-week observation period post dosing, the control animals had tumor growth greater than 2000 m³ while two experimental animals had no tumors and 4 had dramatically smaller tumor volumes than controls. All animals were sacrificed and histopathology of tumor sites was assessed.

The histopathology of tumors was evaluated as follows.

Xenograft tumors (N=11) were presented for histopathologic examination. The tissues were prepared using standard CBI methodology. Tumors were gross trimmed and processed, then embedded in paraffin. Blocks were microtomed at 5 μm and were stained with hematoxylin-eosin. Tissues were examined histopathologically by a board-certified veterinary pathologist. All tissues were in good condition. Minimal to no artifactual changes as a result of tissue handling were present.

The morphology of the tumors was consistent with the xenograft cell line. There were differences between the Control (comprising bacteriostatic water) and the tumors treated with the experimental formulation in that the treated tumors were clearly smaller and had a much larger area of necrosis than that of the control tumors. The individual findings are presented in Table 1 below.

Two of the mice treated with the experimental formulation lost all appearance of tumor and their tissues were not included in the histology study.

The histopathologic findings are shown in table 2-1.

TABLE 2-1
Histopathologic Findings
Histopathologic Findings

| Group | Animal ID | Tumor Size | Intratumoral Necrosis |
|---|---|---|---|
| Control | G1A-1 | large | moderate |
| | G1A-2 | large | mild |
| | G1B-2 | large | mild |
| | G1B-3 | Very large | moderate |
| | G1B-4 | large | moderate |
| Treated | G2A-1 | small | severe |
| | G2A-3 | small | severe |
| | G2B-1 | small | severe |
| | G2B-3 | small | severe |
| | G2B-5 | moderate | severe |

This study shows that tumor cells contacted with the composition of the invention demonstrates increase in intratumoral necrosis compared to a tumor contacted with a control comprising bacteriostatic water.

### Example 3

A study is conducted to evaluate the anti-tumor activity of the components of the transdermal formulation intratumorally against established subcutaneous CT26 murine colon carcinoma in female Balb/c mice.

The test compounds, dosing schedule, and doses are shown in Table 3-1 below. Vehicle control is saline solution. 3-surfactant mixture is a mixture of Nonaethylene glycol monododecyl ether, 1-Methyl-2-pyrrolidinone, and Linoleic Acid.

TABLE 3-1
Compounds,

| | # | | Route of | | Dose |
|---|---|---|---|---|---|
| Group | Animals | Compound | Administration | Schedule | (mg/kg/inj) |
| 1 | 10 | Vehicle Control | IT | QDx7 | 50 μL/ |
| | | | | | injection |
| 2 | 10 | 3-surfactant mixture Group | IT | QDx7 | 50 μL/ |
| | | | | | injection |
| 3 | 10 | Nonaethylene glycol | IT | QDx7 | 50 μL/ |
| | | monododecyl ether | | | injection |
| 4 | 10 | 1-Methyl-2-pyrrolidinone | IT | QDx7 | 50 μL/ |
| | | anhydrous | | | injection |
| 5 | 10 | Linoleic Acid | IT | QDx7 | 50 μL/ |
| | | | | | injection |

Table 3-2 below shows the study parameters.

TABLE 3-2
Study Parameters.
Disease Induction, Staging, Dosing, Observations

| | | | 100-150 mm3 | | |
|---|---|---|---|---|---|
| | | Enrollment | (target mean | | |
| Species | Mouse | Criteria | 125 mm3) | Necropsy | Yes |
| Gender | Female | Est. Staging | 7 Days | Max. In-Life | 60 Days |
| | | Duration | | Duration | |
| Strain | Balb/c | #Study Animals | 50 | Clinical | 7 |
| | | | | Obs/wk | |
| Vendor | Envigo | % Triage | 60% | Weights/wk | 3 |
| Model | CT26.WT | # Total Animals | 80 | Measure/wk | 3 |
| Implant | Cells | Implant Location | SC - axilla | Cells/Implant | 5.00E+05 |
| Type | | | (high) | | |
| Total # | 6.00E+07 | % Excess Cells | 50% | % Matrigel | 0 |
| Cells | | | | | |
| Total # Rad | 0 | Total #Doses | 350 | Total #Drug Preps | 29 |
| Doses | | | | | |
| Acclimation | 7 | Age Range (weeks) | 6-7 | | |
| Duration | | | | | |
| Implant | 200.0 μL | | | | |
| Volume | | | | | |

Tumor cells are implanted subcutaneously (Day 0). Once enrollment criteria are achieved, animals are distributed into treatment groups such that the mean tumor burden in each group is within 10% of the overall mean. Mice are dosed individually by body weight (50 μL fixed/injection intratumorally) on the day of treatment as described above. Animals are dose daily for 7 days beginning on Day 11 and ending on Day 17. Animals are held for tumor growth delay endpoint/tumor growth inhibition endpoint and complete regression/partial regression/tumor free survivor determination. Mean and Median Tumor volumes (mm³) are shown in Table 3-3.

TABLE 3-3
Mean and Median Tumor Volume (mm³) by Group
Vehicle

| | | | | | | | | DT | | | TES |
|---|---|---|---|---|---|---|---|---|---|---|---|
| | | | | | | | | Group | | | Group |
| Mean | 129 | 238 | 442 | 746 | 981 | 1408 | 2257 | Median | 3.6 | 18.0 | Median |
| SE | 4 | 26 | 38 | 44 | 74 | 83 | 195 | | | 0.0 | TGD |
| Median | 126 | 207 | 451 | 750 | 999 | 1455 | 2147 | | | | |

Transdermal Group

| | | | | | | | | DT | | | TES |
|---|---|---|---|---|---|---|---|---|---|---|---|
| | | | | | | | | Group | | | Group |
| Mean | 136 | 296 | 462 | 860 | 1173 | 1385 | 2228 | Median | 3.6 | 17.8 | Median |
| SE | 4 | 17 | 49 | 98 | 154 | 180 | 189 | | | −0.2 | TGD |
| Median | 144 | 279 | 405 | 787 | 1046 | 1568 | 2277 | | | | |

Nonaethylene glycol monododecyl ether

| | | | | | | | | DT | | | TES |
|---|---|---|---|---|---|---|---|---|---|---|---|
| | | | | | | | | Group | | | Group |
| Mean | 126 | 236 | 416 | 761 | 996 | 1364 | 2017 | Median | 3.7 | 17.7 | Median |
| SE | 4 | 19 | 47 | 68 | 100 | 154 | 194 | | | −0.4 | TGD |
| Median | 125 | 246 | 368 | 819 | 988 | 1311 | 2009 | | | | |

1-Methyl-2-pyrrolidinone anhydrous

| | | | | | | | | DT | | | TES |
|---|---|---|---|---|---|---|---|---|---|---|---|
| | | | | | | | | Group | | | Group |
| Mean | 134 | 278 | 428 | 688 | 1008 | 1216 | 1910 | Median | 4.0 | 18.8 | Median |
| SE | 5 | 21 | 40 | 79 | 154 | 133 | 133 | | | 0.8 | TGD |
| Median | 141 | 295 | 426 | 624 | 918 | 1183 | 1812 | | | | |

Linoleic Acid

| | | | | | | | | DT | | | TES |
|---|---|---|---|---|---|---|---|---|---|---|---|
| | | | | | | | | Group | | | Group |
| Mean | 132 | 283 | 481 | 777 | 1291 | 1118 | 2035 | Median | 3.5 | 18.7 | Median |
| SE | 5 | 29 | 64 | 104 | 221 | 134 | 260 | | | 0.6 | TGD |
| Median | 138 | 270 | 411 | 700 | 1030 | 1152 | 2250 | | | | |

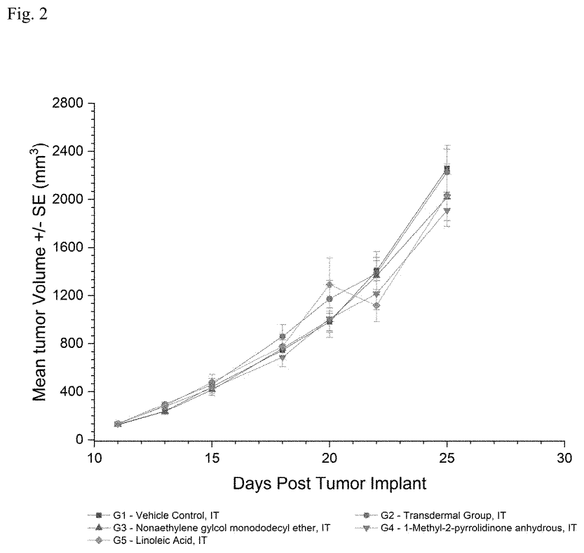

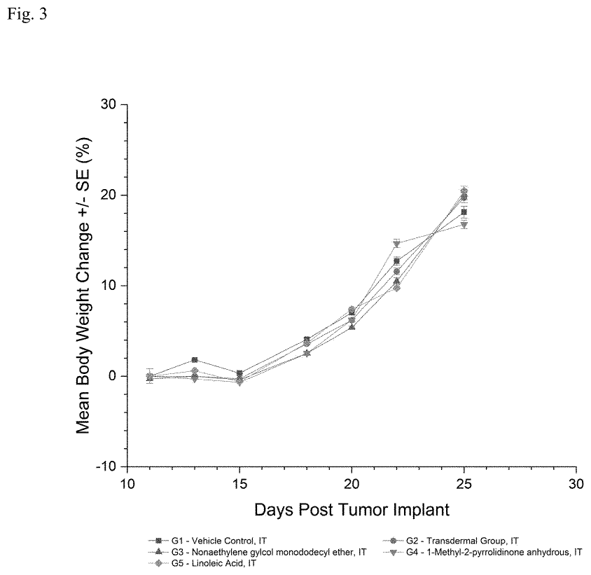

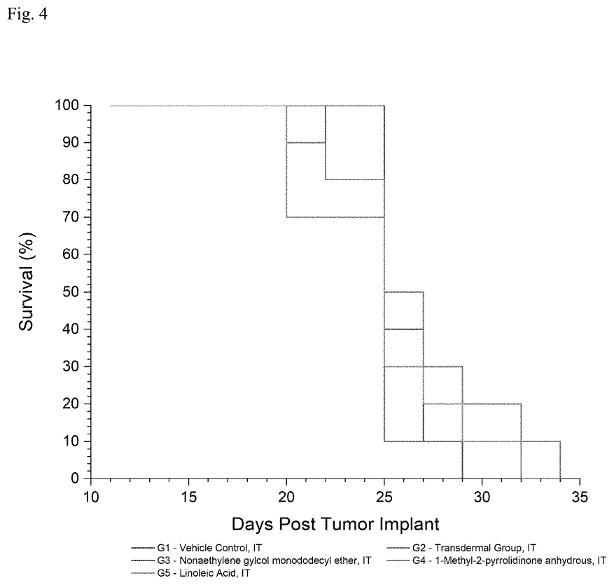

The Study Results are given in FIGS. 2-4.

In some embodiments, the disclosure is directed to the following aspects:

Aspect 1. A method of treating cancer in a patient in need thereof, comprising contacting said patient's cancer cells with an effective amount of a pharmaceutical composition comprising a surfactant and a bile acid or bile acid salt.
Aspect 2. The method of aspect 1, wherein the surfactant is an ionic surfactant, a non-ionic surfactant, an amphoteric surfactant, or a mixture thereof.
Aspect 3. A method of reducing the size of a tumor is a patient in need thereof, comprising contacting said patient's tumor with an effective amount of a pharmaceutical composition comprising a surfactant and a bile acid or bile acid salt.
Aspect 4. The method of any one of aspects 1-3, wherein the surfactant is a compound of formula (I)

R—(OCH₂CH₂)_{y}—OH (I)

wherein R is C₁₋₂₀alkyl, C₂₋₂₀alkenyl; or C₂₋₂₀alkynyl; and y is 1 to 25.
Aspect 5. The method of aspect 3, wherein R is C₁₋₂₀alkyl.
Aspect 6. The method of any one of aspects 4 or 5, wherein y is 5 to 15.
Aspect 7. The method of any one of aspects 1-4, wherein the compound of formula I is cetomacrogol 1000; octadecan-1-ol, ethoxylated; polyoxyethylene(12)tridecyl ether; polyoxyethylene(10)tridecyl ether; fatty alcohol polyoxyethylene ether, polyoxyethylene branched nonylcyclohexyl ether, nonaethylene glycol monododecyl ether, 23-{[4-(2,4,4-trimethyl-2-pentanyl)cyclohexyl]oxy}-3,6,9,12,15,18,21-heptaoxatricosan-1-ol, or a combination thereof.
Aspect 8. The method of aspect 7, wherein the compound of formula I is nonaethylene glycol monododecyl ether.
Aspect 9. The method of aspect 4, wherein R is C₂₋₂₀alkenyl.
Aspect 10. The method of any one of aspect 4 or aspect 9, wherein the compound of formula I is polyoxyl(10)oleyl ether, polyethylene glycol tert-octylphenyl ether, or a combination thereof.
Aspect 11. The method of aspect 4, wherein R is C₂₋₂₀alkynyl.
Aspect 12. The method of any one of aspects 1-3, wherein the surfactant is a tetrafunctional block copolymer surfactant terminating in primary hydroxyl groups.
Aspect 13. The method of aspect 12, wherein the tetrafunctional block copolymer surfactant terminating in primary hydroxyl groups is ethylenediaminetetrakis(ethoxylate-Block-propoxylate).
Aspect 14. The method of any one of aspects 1-3, wherein the surfactant is a sorbitan derivative.
Aspect 15. The method of aspect 14, wherein the sorbitan derivative is polyoxyethylene sorbitan tetraoleate, 1,4-anhydro-6-O-palmitoyl-D-glucitol (sorbitan, monohexadecanoate), a polyethylene glycol sorbitan monolaurate, or a combination thereof.
Aspect 16. The method of any one of aspects 1-3, wherein the surfactant is a C₈₋₁₀alkyl ammonium salt.
Aspect 17. The method of aspect 16, wherein the C₈₋₁₀alkyl ammonium salt is methyltrialkyl(C₈-C₁₀)ammonium chloride (ADOGEN 464).
Aspect 18. The method of any one of aspects 1-3, wherein the surfactant is the compound of formula II:

HO—(CH₂CH₂)ₘ—C(CH₃)(C₄H₉)—C≡C—C(CH₃)(C₄H₉)—(OCH₂CH₂)ₙ—OH (II)

wherein m and n are each independently 1 to 25.
Aspect 19. The method of any one of aspects 1-3, wherein the surfactant is a compound of formula III:

R²—N(R¹)—C(O)—R³ (III)

wherein each R¹ is independently H or C₁₋₃alkyl; and R² and R³ are independently C₁₋₇alkyl or together with the atoms to which they are attached, form a lactam having 3 to 10 carbon atoms.
Aspect 20. The method of aspect 19, wherein R¹ is methyl, ethyl, or propyl.
Aspect 21. The method of any one of aspects 19 or 20, wherein R² and R³, together with the atoms to which they are attached, form a lactam having 3 to 10 carbon atoms.
Aspect 22. The method of aspect 19, wherein the lactam is a pyrrolidone.
Aspect 23. The method of aspect 22, wherein the pyrrolidone is 1-methyl-2-pyrrolidinone.
Aspect 24. The method of any one of aspects 1-3, wherein the surfactant is an organic acid that is not a bile acid.
Aspect 25. The method of aspect 24, wherein the organic acid that is not a bile acid is a fatty acid or a C₁₋₆alkyl acid.
Aspect 26. The method of aspect 25, wherein the fatty acid is linoleic acid.
Aspect 27. The method of any one of aspects 1-26, wherein the bile acid is deoxycholic acid, cholic acid, glycocholic acid, taurocholic acid, tauroursodeoxycholic acid, chenodeoxycholic acid, glycochenodeoxycholic acid, taurochenodeoxycholic acid, or lithocholic acid.
Aspect 28. The method of aspect 27, wherein the bile acid is tauroursodeoxycholic acid.
Aspect 29. The method of any one of aspects 1-26, wherein the bile acid salt is a salt of deoxycholic acid, cholic acid, glycocholic acid, taurocholic acid, tauroursodeoxycholic acid, chenodeoxycholic acid, glycochenodeoxycholic acid, taurochenodeoxycholic acid, or lithocholic acid.
Aspect 30. The method of aspect 29, wherein the bile acid salt is a salt of tauroursodeoxycholic acid.
Aspect 31. The method of any one of aspects 1-30, wherein the pharmaceutical composition further comprises a sulfoxide.
Aspect 32. The method of any one of aspects 1-31, wherein the pharmaceutical composition further comprises a urea.
Aspect 33. The method of any one of aspects 1-32, wherein the pharmaceutical composition further comprises ethyl acetate.
Aspect 34. The method of any one of aspects 1-33, wherein the pharmaceutical composition further comprises a C₁₋₁₀alkyl alcohol.
Aspect 35. The method of aspect 34 wherein the C₁₋₁₀alkyl alcohol is glycerol, propylene glycol, methanol, ethanol, isopropanol, 1-propanol, butanol, t-butanol, pentanol, 1-octanol, benzyl alcohol, or a combination thereof.
Aspect 36. The method of any one of aspects 1-3, wherein the pharmaceutical composition comprises nonaethylene glycol monododecyl ether, 1-methyl-2-pyrrolidinone, linoleic acid, and a bile acid or bile acid salt.
Aspect 37. The method of any one of aspects 1-36, wherein the pharmaceutical composition is in the form of a solution, a suspension, a gel, an emulsion, or a dispersion.
Aspect 38. The method of any one of aspects 1-37, further comprising administering to said patient a second therapeutic agent.
Aspect 39. The method of aspect 38, wherein the second therapeutic agent is an anticancer agent.
Aspect 40. The method of any one of aspects 1-3, wherein the pharmaceutical composition comprises nonaethylene glycol monododecyl ether, l-methyl-2-pyrrollidinone, and a bile acid or bile acid salt.
Aspect 41. The method of aspect 40, wherein the bile acid is tauroursodeoxycholic acid.
Aspect 42. The method of aspect 40, wherein the bile acid salt is sodium deoxycholate.
Aspect 43. The method of any one of aspects 40-42, wherein the pharmaceutical composition further comprises linoleic acid.
Aspect 44. The method of any one of aspects 40-43, wherein the pharmaceutical composition further comprises benzyl alcohol.

What is claimed:

1. A method of treating cancer in a patient in need thereof, comprising contacting said patient's cancer cells with an effective amount of a pharmaceutical composition comprising a surfactant and a bile acid or bile acid salt.

2. The method of claim 1, wherein the surfactant is an ionic surfactant, a non-ionic surfactant, an amphoteric surfactant, or a mixture thereof.

3. A method of reducing the size of a tumor is a patient in need thereof, comprising contacting said patient's tumor with an effective amount of a pharmaceutical composition comprising a surfactant and a bile acid or bile acid salt.

4. The method of claim 1, wherein the surfactant is a compound of formula (I):
R—(OCH₂CH₂)_{y}—OH (I)
wherein R is C₁₋₂₀alkyl, C₂₋₂₀alkenyl; or C₂₋₂₀alkynyl; and y is 1 to 25.

5. The method of claim 4, wherein R is C₁₋₂₀alkyl.

6. The method of claim 4, wherein y is 5 to 15.

7. The method of claim 4, wherein the compound of formula I is cetomacrogol 1000; octadecan-1-ol, ethoxylated; polyoxyethylene(12)tridecyl ether; polyoxyethylene(10)tridecyl ether; fatty alcohol polyoxyethylene ether, polyoxyethylene branched nonylcyclohexyl ether, nonaethylene glycol monododecyl ether, 23-{[4-(2,4,4-trimethyl-2-pentanyl)cyclohexyl]oxy}-3,6,9,12,15,18,21-heptaoxatricosan-1-ol, or a combination thereof.

8. The method of claim 7, wherein the compound of formula I is nonaethylene glycol monododecyl ether.

9. The method of claim 4, wherein R is C₂₋₂₀alkenyl.

10. The method of claim 4, wherein the compound of formula I is polyoxyl(10)oleyl ether, polyethylene glycol tert-octylphenyl ether, or a combination thereof.

11. The method of claim 4, wherein R is C₂₋₂₀alkynyl.

12. The method of claim 1, wherein the surfactant is a tetrafunctional block copolymer surfactant terminating in primary hydroxyl groups.

13. The method of claim 12, wherein the tetrafunctional block copolymer surfactant terminating in primary hydroxyl groups is ethylenediaminetetrakis(ethoxylate-Block-propoxylate).

14. The method of claim 1, wherein the surfactant is a sorbitan derivative.

15. The method of claim 14, wherein the sorbitan derivative is polyoxyethylene sorbitan tetraoleate, 1,4-anhydro-6-O-palmitoyl-D-glucitol (sorbitan, monohexadecanoate), a polyethylene glycol sorbitan monolaurate, or a combination thereof.

16. The method of claim 1, wherein the surfactant is a C₈₋₁₀alkyl ammonium salt.

17. The method of claim 16, wherein the C₈₋₁₀alkyl ammonium salt is methyltrialkyl(C₈-C₁₀)ammonium chloride (ADOGEN 464).

18. The method of claim 1, wherein the surfactant is the compound of formula II:
HO—(CH₂CH₂O)ₘ—C(CH₃)(C₄H₉)—C≡C—C(CH₃)(C₄H₉)—(OCH₂CH₂)ₙ—OH (II)
wherein m and n are each independently 1 to 25.

19. The method of claim 1, wherein the surfactant is a compound of formula III:
R²—N(R′)—C(O)—R³ (III)
wherein each R¹ is independently H or C₁₋₃alkyl; and R² and R³ are independently C₁₋₇alkyl or together with the atoms to which they are attached, form a lactam having 3 to 10 carbon atoms.

20. The method of claim 19, wherein R¹ is methyl, ethyl, or propyl.

21. The method of claim 19, wherein R² and R³, together with the atoms to which they are attached, form a lactam having 3 to 10 carbon atoms.

22. The method of claim 19, wherein the lactam is a pyrrolidone.

23. The method of claim 22, wherein the pyrrolidone is 1-methyl-2-pyrrolidinone.

24. The method of claim 1, wherein the surfactant is an organic acid that is not a bile acid.

25. The method of claim 24, wherein the organic acid that is not a bile acid is a fatty acid or a C₁-6alkyl acid.

26. The method of claim 25, wherein the fatty acid is linoleic acid.

27. The method of claim 1, wherein the bile acid is deoxycholic acid, cholic acid, glycocholic acid, taurocholic acid, tauroursodeoxycholic acid, chenodeoxycholic acid, glycochenodeoxycholic acid, taurochenodeoxycholic acid, or lithocholic acid.

28. The method of claim 27, wherein the bile acid is tauroursodeoxycholic acid.

29. The method of claim 1, wherein the bile acid salt is a salt of deoxycholic acid, cholic acid, glycocholic acid, taurocholic acid, tauroursodeoxycholic acid, chenodeoxycholic acid, glycochenodeoxycholic acid, taurochenodeoxycholic acid, or lithocholic acid.

30. The method of claim 29, wherein the bile acid salt is a salt of tauroursodeoxycholic acid.

31. The method of claim 1, wherein the pharmaceutical composition further comprises a sulfoxide.

32. The method of claim 1, wherein the pharmaceutical composition further comprises a urea.

33. The method of claim 1, wherein the pharmaceutical composition further comprises ethyl acetate.

34. The method of claim 1, wherein the pharmaceutical composition further comprises a C₁₋₁₀alkyl alcohol.

35. The method of claim 34 wherein the C₁₋₁₀alkyl alcohol is glycerol, propylene glycol, methanol, ethanol, isopropanol, 1-propanol, butanol, t-butanol, pentanol, 1-octanol, benzyl alcohol, or a combination thereof.

36. The method of claim 1, wherein the pharmaceutical composition comprises nonaethylene glycol monododecyl ether, 1-methyl-2-pyrrolidinone, linoleic acid, and a bile acid or bile acid salt.

37. The method of claim 1, wherein the pharmaceutical composition is in the form of a solution, a suspension, a gel, an emulsion, or a dispersion.

38. The method of claim 1, further comprising administering to said patient a second therapeutic agent.

39. The method of claim 38, wherein the second therapeutic agent is an anticancer agent.

40. The method of claim 1, wherein the pharmaceutical composition comprises nonaethylene glycol monododecyl ether, l-methyl-2-pyrrollidinone, and a bile acid or bile acid salt.

41. The method of claim 40, wherein the bile acid is tauroursodeoxycholic acid.

42. The method of claim 40, wherein the bile acid salt is sodium deoxycholate.

43. The method of claim 40, wherein the pharmaceutical composition further comprises linoleic acid.

44. The method of claim 40, wherein the pharmaceutical composition further comprises benzyl alcohol.
